(19) United States
(12) Patent Application Publication
(10) Pub. No.: US 2025/0339393 A1
(43) Pub. Date: Nov. 6, 2025

(54) USE OF TRANSMEMBRANE PH GRADIENT LIPOSOMES FOR TREATING HYPERAMMONEMIC CRISIS ASSOCIATED WITH INBORN ERRORS OF METABOLISM

(71) Applicants: GENFIT, LOOS (FR); VERSANTIS AG, Zurich (CH)

(72) Inventors: Vincent Forster, Zurich (CH); Meriam Kabbaj, Zurich (CH)

(21) Appl. No.: 19/128,345

(22) PCT Filed: Nov. 15, 2023
(86) PCT No.: PCT/EP2023/081900

§ 371 Date: May 8, 2025

(30) Foreign Application Priority Data
Nov. 16, 2022 (EP) 22306686.1

(51) Int. Cl. A61K 31/194; A61K 9/1271; A61K 47/02; A61K 47/26

(52) U.S. Cl. CPC A61K 31/194; A61K 9/1271; A61K 47/02; A61K 47/26

(57) ABSTRACT

The present disclosure provides a use of a liposomal suspension comprising transmembrane pH-gradient liposomes, for the treatment of an acute hyperammonemia episode (HAC) associated with inborn errors of metabolism (IEM) in a subject, wherein the treatment comprises intraperitoneally administering the liposomal suspension to the subject and removing a dialysate containing ammonia-loaded liposomes from the subject.

### FIELD OF THE DISCLOSURE

The present disclosure relates to the use of transmembrane pH gradient liposomes for treating hyperammonemic crisis associated with inborn errors of metabolism in a subject in need thereof. More specifically, the subject in need thereof has a urea cycle disorder or an organic acidemia.

### BACKGROUND OF THE DISCLOSURE

Hyperammonemic crisis (HAC) is manifested by acute hyperammonemia, which is a life-threatening condition that represents a substantial cause of brain damage and death if not treated early and effectively [Savy 2018].

HAC can be associated with inborn errors of metabolism (IEM) or with acquired clinical conditions that are distinct from IEM, based on their pathogenesis, clinical presentation and treatment. In IEM, the HAC together with its direct neurological consequences is the primary issue, while in most other clinical presentations hyperammonemia is driven by liver failure, other organ dysfunctions and can be drug-induced.

More particularly, HAC associated with IEM is linked to inherited defects in genes encoding for enzymes affecting the urea cycle (the physiological elimination of ammonia) either directly in urea cycle disorders (UCDs) (primary hyperammonemia) or indirectly in organic acidemia (OA) (secondary hyperammonemia). The entire urea cycle is only present in the liver where it is expressed in periportal hepatocytes; the liver is otherwise healthy.

The biological and clinical manifestations, the outcome, and the prognosis of the disease in IEM subjects are all solely driven by the genetic defect leading to hyperammonemia. Indeed, coma duration and levels of blood ammonia concentrations are the principal factors for determining mortality and neurologic outcome. Ammonia levels are the unique driver of the treatment decision tree.

The other clinical conditions causing HAC are all acquired diseases and conditions, 95.7% of which are related to liver diseases. In liver diseases, hyperammonemia is due to a decreased ammonia elimination occurring when the number of functional periportal hepatocytes fall below a threshold required for sufficient ammonia detoxification. Neurologic manifestations of liver failure are called “hepatic encephalopathy”. In contrast to hyperammonemia in IEM, hepatic encephalopathy pathogenesis is thought to involve multiple factors including the role of neurotoxins other than ammonia, impaired neurotransmission due to metabolic changes in liver failure, changes in brain energy metabolism, systemic inflammatory response and alterations of the blood brain barrier]. In liver failure, the clinical manifestations, prognosis and treatment of the disease are mainly driven by the destruction of the liver itself, ultimately inducing multiorgan failures. Treatment of hepatic encephalopathy in chronic liver diseases relies on controlling the precipitating factors and reducing the gut ammonia production with lactulose with or without rifaximin to prevent relapses. The remaining 4.3% non-hepatic causes of acquired hyperammonemia are rare and are associated with severe conditions leading to an increased production of ammonia. In these non-hepatic conditions, the clinical manifestations, the outcome and the prognosis of the diseases involve multiorgan dysfunction associated with states of increased catabolism such as in hemato-oncological disorders, organ transplantation, and severe gastrointestinal infections. Drug-induced hyperammonemia can also result from interference with the urea cycle or enhancement of renal release of ammonia into the systemic circulation. Valproic acid is the most well-known causative agent. Treatment of non-liver disease acquired hyperammonemia is mainly based on removal of the causal event and organ support, such as hemodialysis.

In conclusion, HAC in IEM represents a unique distinct condition involving a genetic disorder affecting the metabolism of ammonia by the urea cycle in an otherwise healthy liver.

The prevalence of these genetic disorders may be higher than current estimates (1/35,000-1/69,000 births considering all UCDs) due to a lack of reliability in screening of newborns as well as under-diagnosis of deceased cases. UCDs represent 23% of HAC in children admitted in pediatric intensive care. The prevalence of all underlying genetic disorders leading to HACs lies below the threshold of 200,000 persons in the United States.

Current research targets the correction of the defect underlying each IEM causing HAC; however, the results are so far inconclusive. Trials of gene transfer for deficiency in ornithine transcarbamylase (further described below) using an adenoviral vector did not result in a useful increase in enzyme activity [Leonard 2004]. These studies were discontinued because of severe complications and the death of one patient [Raper 2003]. Gene transfer in a bovine model of citrullinemia showed positive results but it is yet to be tested in man [Leonard 2004]. There is an ongoing open-label clinical trial in adults with late-onset OTCD (ClinicalTrials.gov Identifier: NCT02991144). Hepatocyte transfusion is an alternative therapy that is currently under investigation. Hepatocytes infused into the liver may provide an alternative source of enzyme. However, any biochemical correction to date appears to have been transient and the patients' benefit remains unclear [Horslen 2003].

The clinical manifestation of HAC is similar in all IEMs, regardless of their specific underlying genetic defects (further defined below); the elevated ammonia blood level is solely responsible for the neurological clinical manifestations and does not involve liver failure as a cause of hyperammonemia. In light of the similar clinical presentation, common treatment and outcome if untreated, regardless of the underlying genetic defects, HAC associated with IEM is identified as a single condition with a high unmet medical need.

The current initial management of HAC does not take into account the specific diagnosis of the genetic disorder, which usually takes several days; the medical objective is uniformly to rapidly reduce ammonia levels. To reduce high mortality and morbidity associated with HACs in IEM, immediate start of treatment is thought to reduce mortality and morbidity [Enns 2007; Hediger 2018; Savy 2018]. Treatment involves dietary measures, ammonium scavengers and immediate transfer of the patient to a tertiary center for emergency detoxification of ammonia by acute renal replacement therapy (RRT), the fastest way to clear ammonium from the bloodstream.

Current treatments of HAC caused by an IEM in neonates specifically, include hemodialysis (HD), peritoneal dialysis (PD), or continuous RRT to decrease plasma levels. Although PD can be started immediately, it has a slower rate of ammonia removal making it difficult to balance ammonia formation. Although HD is the most efficient way to remove ammonia, it is often unavailable, highly invasive, hypotension is a frequent complication and ammonia level tends to rebound after HD is terminated. CRRT, and more specifically continuous venovenous HD with a high dialysate flow rate, appears to be the best available option. In practice, pediatric patients presenting HAC must be transferred in highly specialized tertiary centers having CRRT devices adapted to their size.

Drawbacks of CRRT in neonates include difficulty in establishing vascular access lines, difficulty in fluid balance as well as a lack of equipment appropriate for neonates. The relatively large circuit volume (60 mL) needed for blood priming carries several risks. A detailed fluid balance is not possible, and the current instruments used for CRRT are not approved or cleared for babies weighing less than 8 kg. Consequently, dialysis in IEM HAC is often initiated late when ammonia levels are above 1000 μmol/L and this may contribute to poor outcomes [Hediger 2018]. There is no acute treatment available for early onset crises.

Additionally, the significant proportion of late onset HACs (7% previously reported in 299 patients representing 1181 episodes of acute HA) still need rapid dialysis to avoid further impairment of the neurological function [Enns 2007].

Alternative therapeutic interventions for the treatment of HAC associated with IEM, and in particular for the treatment of pediatric patients (e.g., neonates) presenting HAC associated with IEM need to be developed.

The present description refers to a number of documents, the content of which is herein incorporated by reference in their entirety.

### SUMMARY OF THE DISCLOSURE

The present disclosure provides an alternative method of treating HAC associated with IEM. It provides liposomes conferring optimized ammonia clearance to peritoneal fluid, allowing initiation of efficient PD immediately after HAC is confirmed and before transferring the patient to a tertiary center. Any patient affected by this disease may benefit from the method of presented herein, but it is particularly useful in in pediatric patients. The treatment provided by the present disclosure reduces burden on the patients and parents and decreases healthcare system costs.

More specifically, in accordance with the present disclosure, there are provided the following items:

Item 1. Use of a liposomal suspension comprising transmembrane pH-gradient liposomes, for the treatment of an acute hyperammonemia episode (HAC) associated with inborn errors of metabolism (IEM) in a subject, wherein the treatment comprises intraperitoneally administering the liposomal suspension to the subject and removing a dialysate containing ammonia-loaded liposomes from the subject.

Item 2. The use of item 1, wherein the subject is a pediatric subject.

Item 3. The use of item any one of items 1 to 3, wherein the subject has a urea cycle disorder (UCD).

Item 4. The use of item 3, wherein the UCD is ornithine transcarbamylase deficiency.

Item 5. The use of any one of items 1 to 5, wherein the liposomes contain a hydroxy acid, preferably citric acid, most preferably citric acid anhydrous.

Item 6. The use of item 5, wherein the liposomes contain about 200 nM citric acid anhydrous, an preferably an internal pH of about 2

Item 7. The use of any one of items 1 to 6, wherein the liposomes' lipid bilayer comprises at least one phospholipid as main constituent.

Item 8. The use of item 7, wherein the at least one phospholipid comprises dipalmitoylphosphatidylcholine (DPPC), preferably in a range of 60 mol % to 90 mol %.

Item 9. The use of any one of items 1 to 8, wherein the liposomes' lipid bilayer comprises cholesterol, preferably in a range of 10 to 40 mol %.

Item 10. The use of item 9, wherein the liposomes' lipid bilayer further comprises 1,2-distearoyl-sn-glycero-3-phosphoethanol-amine-N-[methoxy(PEG)-2000] (DSPE-PEG), preferably in a range of 0.2 to 5 mol %.

Item 11. The use of any one of items 1 to 6, wherein the bilayer of the liposomes contains dipalmitoylphosphatidylcholine (DPPC), cholesterol and 1,2-distearoyl-sn-glycero-3-phosphoethanol-amine-N-[methoxy(PEG)-2000] (DSPE-PEG) at 85.5:14:0.5 mol %, and the liposomes' inner compartment contains citric acid anhydrous.

Item 12. The use of any one of items 1 to 11, wherein the liposomes have an average diameter between about 8 μm and 12 μm.

Item 13. The use of any one of items 1 to 12, wherein the liposomal suspension contains (i) xylitol, (ii) sodium chloride, (iii) sodium hydroxide, (iv) potassium chloride, (v) calcium chloride or (vii) any combination of at least two of (i) to (v), preferably the combination comprises (i) to (v),

### Definitions

### Liposomes

### Liposomes Composition

Liposomes according to the present disclosure comprise a lipid bilayer membrane enclosing an acidic buffer (acidic solution).

### Lipid Bilayer Membrane

In preferred embodiments, the liposome lipid bilayer membrane comprises at least one natural or synthetic phospholipid. Preferred phospholipids are long saturated phospholipids, e.g., those having alkyl chains of more than 12, preferably more than 14, more preferably more than 16, most preferably more than 18 carbon atoms.

In specific embodiments, the natural or synthetic phospholipid comprises at least one of 1,2-Dilauroyl-sn-Glycero-3-Phosphocholine (DLPC); 1,2-Dimyristoyl-sn-Glycero-3-Phosphocholine (DMPC); 1,2-Dipalmitoyl-sn-Glycero-3-Phosphocholine (DPPC); 1,2-Distearoyl-sn-Glycero-3-Phosphocholine (DSPC); 1,2-Dioleoyl-sn-Glycero-3-Phosphocholine (DOPC); 1,2-Dimyristoyl-sn-Glycero-3-Phosphoelhanolamine (DMPE); 1,2-Dipalmitoyl-sn-Glycero-3-Phosphoelhanolamine (DPPE); 1,2-Distearoyl-sn-Glycero-3-Phosphoelhanolamine (DSPE); 1,2-Dioleoyl-sn-Glycero-3-Phosphoelhanolamine (DOPE); 1-Myristoyl-2-Palmitoyl-sn-Glycero-3-Phosphocholine (MPPC); 1-Palmitoyl-2-Myristoyl-sn-Glycero-3-Phosphocholine (PMPC); 1-Stearoyl-2-Palmitoyl-sn-Glycero-3 Phosphocholine (SPPC); 1-Palmitoyl-2-Stearoyl-sn-Glycero-3-Phosphocholine (PSPC); 1,2-Dimyristoyl-sn-Glycero-3-[Phospho-rac-(1-glycerol)] (DMPG); 1,2-Dipalmitoyl-sn-Glycero-3-[Phospho-rac-(1-glycerol)] (DPPG); 1,2-Distearoyl-sn-Glycero-3-[Phospho-rac-(1-glycerol)] (DSPG); 1,2-Dioleoyl-sn-Glycero-3-[Phospho-rac-(1-glycerol)] (DOPG); 1,2-Dimyristoyl-sn-Glycero-3-Phosphate (DMPA); 1,2-Dipalmitoyl-sn-Glycero-3-Phosphate (DPPA); 1,2-Dipalmitoyl-sn-Glycero-3-[Phospho-L-Serine] (DPPS); natural L-α-phosphatidylcholine (from chicken egg, EPC, or from soy, SPC). In specific embodiments, the natural or synthetic phospholipid is DPPC. In specific embodiments, the main constituent of the liposome lipid bilayer is the at least one natural or synthetic phospholipid. In specific embodiments, the at least one natural or synthetic phospholipid forms at least 60 mol %, 65 mol %, 70 mol %, 75 mol %, 80 mol %, or 85 mol % of the liposome bilayer membrane. In specific embodiments, the natural or synthetic phospholipid forms about 85.5 mol % of the liposome bilayer membrane.

In other embodiments, the liposome lipid bilayer membrane further comprises an ammonia retention-enhancing compound. In specific embodiments, the ammonia retention-enhancing compound comprises a sterol derivative. In other specific embodiments, the sterol derivative is cholesterol. In specific embodiments, the at least one ammonia retention-enhancing compound forms at least 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, or 50 mol % of the liposome bilayer membrane. In specific embodiments, the at least one ammonia retention-enhancing compound forms at least 10 mol % of the liposome bilayer membrane. In specific embodiments, the at least one ammonia retention-enhancing compound forms about 14% of the liposome bilayer membrane.

In other embodiments, the liposome lipid bilayer membrane further comprises at least one steric stabilizer, such as at least one PEGylated compounds, preferably at least one PEGylated lipid, more preferably DSPE-PEG. In specific embodiments, the at least one steric stabilizer forms at least 0.1 mol %, 0.2 mol %, 0.3 mol %, 0.4 mol %, 0.5 mol %, 0.6 mol %, 0.7 mol %, 0.8 mol %, 0.9 mol %, 1 mol %, 1.5 mol %, 2 mol %, 2.5 mol %, 3 mol %, 3.5 mol %, 4 mol %, 4.5 mol %, 5 mol %, 5.5 mol %, 6 mol %, 6.5 mol %, 7 mol %, 7.5 mol %, 8 mol %, 8.5 mol %, 9 mol %, 9.5 mol %, or 10 mol % of the liposome bilayer membrane. In specific embodiments, the at least one steric stabilizer forms about 0.5% of the liposome bilayer membrane.

In other embodiments, the liposome lipid bilayer membrane comprises 10 to 100 mol %, more preferably 25 to 75 mol %, more preferably 40 to 70 mol %, most preferably 50 to 60 mol % of at least one sphingolipid, preferably sphingomyelin.

In other embodiments, the liposome lipid bilayer membrane comprises 30 to 100, more preferably 40 to 95, most preferably 45 to 60 mol % of at least one surfactant. In specific embodiments, the at least one surfactant comprises hydrophobic alkyl ether (e.g., Brij), alkyl ester, polysorbate, sorbitan ester, and/or alkyl amide.

In other embodiments, the average diameter size of the liposomes is larger than 900 nm, larger than 1000 nm, larger than 2000 nm, larger than 3000 nm; larger than 4000 nm; larger than 5000 nm, larger than 6000 nm; larger than 7000 nm; between 3000 nm and 15 μm, between 4000 nm and 15 μm, between 5000 nm and 15 μm, between 6000 nm and 15 μm, between 7000 nm and 15 μm, between 8000 nm and 15 μm, between 3000 nm and 14 μm, between 4000 nm and 14 μm, between 5000 nm and 14 μm, between 6000 nm and 14 μm, between 7000 nm and 14 μm, between 8000 nm and 14 μm, between 3000 nm and 13 μm, between 4000 nm and 13 μm, between 5000 nm and 13 μm, between 6000 nm and 13 μm, between 7000 nm and 13 μm, between 8000 nm and 13 μm, to avoid too rapid drainage from the peritoneal space. In specific embodiments, average diameter size of the liposomes is between about 8 μm and about 12 μm.

### Acidic Buffer/Acidic Solution

The acidic buffer in the inner compartment of the liposomes preferably has a high buffering capacity at low pH for a high retention of basic compounds (e.g., ammonia). The acid is not toxic to animals, and does not (or only weakly) permeate out of the liposome membrane.

Without being so limited, the acid enclosed in the liposomes core is (i) a hydroxy acid such as citric acid, isocitric acid, malic acid, tartaric acid, or lactic acid; (ii) a small chain fatty acid such as acetic acid; (iii) a sugar acid such as uronic acid; (iv) a dicarboxylic acid such as malonic acid; (v) a tricarboxylic acid such as propane-1,2,3-tricarboxylic acid or aconitic acid; (vi) a tetracarboxylic acid such as 1,2,3,4-butanetetracarboxylic acid; (vii) a pentacarboxylic acid such as 1,2,3,4,5-pentanepentacarboxylic acid; (viii) a polymeric poly(carboxylic acid) such as poly(acrylic acid) or poly(methacrylic acid); (ix) a polyaminocarboxylic acid such as ethylenediaminetetraacetic acid; or (x) a combination of at least two thereof. In specific embodiments, the acid is a hydroxy acid such as citric acid (e.g., citric acid anhydrous).

In specific embodiments, the concentration of acid used in the method such as the osmotic shock method, may be varied between 50 and 1000 mM. When a hydroxy acid such as citric acid is used, a citric acid solution of between about 100 mM and 900 mM or between about 100 mM and 900 mM, or between about 300 mM and 800 mM, or between about 400 mM and 750 mM, or between about 500 mM and 750 mM, or between about 500 mM and 650 mM or about 600 mM is optimally used; at an osmolality between 500 and 1500 mOsmol/kg, or between 600 and 1400 mOsmol/kg, or between 700 and 1400 mOsmol/kg, between 800 and 1400 mOsmol/kg, or between 800 and 1350 mOsmol/kg, or between 900 and 1350 mOsmol/kg, or between 950 and 1300 mOsmol/kg, or between 950 and 1250 mOsmol/kg, or between 1000 and 1200 mOsmol/kg is optimally used. In another specific embodiment, the concentration of citric acid (e.g., anhydrous) used in the method may be varied between 50 and 1000 mM. When a hydroxy acid such as citric acid is used, a citric acid solution of between about 600 mM is used with an osmolality of between 1000 and 1200 mOsmol/kg is used in the osmotic shock method. In a preferred embodiment, transmembrane pH-gradient liposomes produced by methods described herein have an inner concentration of citric acid anhydrous of about 200 nM, and an inner osmolarity that is physiological i.e., around 350 mOsm/kg.

The acid within the core (inner compartment of liposomes) is present in a concentration that produces a pH between 1 and 6 In the core of the liposomes, and in a specific embodiment, a pH between 1.5 and 3, and in a more specific embodiment, a pH of about 2.

In a specific embodiment, the liposomes contain in their internal compartment/core between 200 nM citric acid (anhydrous), and this core has a pH of about 2.

In alternative embodiments, liposomes for use in the present disclosure are as described in EP 2 882 421 to Leroux et al.

### Composition

In accordance with another aspect of the present invention, there is provided a composition (in the form of a suspension or otherwise) comprising the liposomes of the present disclosure, and at least one pharmaceutically acceptable excipient or carrier. The compositions of the invention can contain a pharmaceutically acceptable carrier/excipient including, without limitation, aqueous or non-aqueous solutions. Pharmaceutically acceptable carriers also can include physiologically acceptable aqueous vehicles (e.g., sugar solutions, saline), neutralizing species (basic or acidic, such as weak bases or weak acids) but also chemical agents used to adjust the osmolarity and/or provide a physiological function. Without being limited excipients encompassed by the present disclosure include glycerol, tris((hydroxymethyl)aminomethane) (TRIS), agents to counteract potential anticoagulant effects of certain weak acids (e.g., citric acid) such as calcium salts (e.g., calcium chloride); other salts such as sodium salts (e.g., sodium chloride), magnesium salts, lactate salts, potassium salts (e.g., potassium chloride); hydroxides (e.g., sodium hydroxide); sugars or polysaccharides (icodextrin, glucose, sorbitol, fructose); amino acids; sugar alcohols (e.g., xylitol, glycerol) or other known carriers/excipients appropriate for the intraperitoneal route. In specific embodiments, the liposomal composition (e.g., suspension) comprise (i) xylitol, (ii) sodium chloride, (iii) sodium hydroxide, (iv) potassium chloride, (v) calcium chloride or (vii) any combination of at least two of (i) to (v), preferably the combination comprises all of (i) to (v).

### Method of Preparing Liposomes

### Osmotic Shock Method

In specific embodiments, a lipid blend can be prepared by mixing the lipid bilayer components in a solvent such as an alcohol or a mixture of water and of an organic solvent (e.g., alcohol such as ethanol or t-butanol), until complete dissolution to form a homogenous lipid mix. The mix can be conducted at room temperature (i.e., around 20-25° C.) or while heating (e.g., at a temperature up to 60° C., preferably up to 45° C.) and optionally slowly mixing.

The mix can optionally be filtered (e.g., 0.2 μm filter). The organic solvent is then removed e.g., by lyophilization, spray drying (e.g., using liquid nitrogen as drying gas), rotary evaporation or otherwise.

The resulting dried lipid blend can then be hydrated in the aqueous medium as further described below.

### Aqueous Medium

In a preferred embodiment, the lipid bilayer components can be directly mixed in an aqueous medium having an osmolarity of not more than 400 mOsm/l (direct lipid hydration method).

In an embodiment, the aqueous medium has a pH value of around 7, e.g., in the range of 6.0 to 7.5, of 6.1 to 7.4, of 6.2 to 7.3, of 6.3 to 7.2, of 6.4 to 7.1, of 6.5 to 7.3, of 6.6 to 7.3, of 6.7 to 7.3, of 6.8 to 7.3, of 6.9 to 7.1, of 6.95 to 7.01, or of about 7.0. In an embodiment, the aqueous medium is chosen from the group consisting of water (e.g., distilled water, deionized water, ultra-pure water or any other kind of purified water), a mixture of water as defined above and organic solvent (e.g., alcohol), aqueous solutions of organic salts, aqueous solutions of inorganic salts, aqueous solutions of organic substances, and combinations thereof. In an embodiment, the aqueous medium is chosen from the group consisting of aqueous solutions of organic salts having a pH value of around 7, aqueous solutions of inorganic salts having a pH value of around 7, aqueous solutions of organic substances having a pH value of around 7, water and combinations thereof.

When using organic or inorganic salts or other organic compounds, these salts or compounds are present in the aqueous medium, in an embodiment, in a low concentration so as to keep a difference in osmolarity between the aqueous medium and the hyperosmotic buffer provoking the osmotic shock which difference is large enough to induce the diffusion of the acidic or basic hyperosmotic buffer into the vesicle internal compartment.

The aqueous medium is a medium that resembles water (in particular with respect to pH) but that might contain a low concentration of salts or compounds, e.g., for buffering the pH value in a neutral range.

A indicated above, the aqueous medium has an osmolarity of not more than 400 mOsm/l. In an embodiment, the osmolarity of the aqueous medium is equal to or less than 300 mOsm/l, equal to or less than 250 mOsm/l, equal to or less than 200 mOsm/l, equal to or less than 150 mOsm/l, equal to or less than 100 mOsm/l, equal to or less than 75 mOsm/l, equal to or less than 50 mOsm/l, equal to or less than 25 mOsm/l, equal to or less than 10 mOsm/l, equal to or less than 5 mOsm/l equal to or less than 1 mOsm/l. In an embodiment, the osmolarity is in the range of 1 mOsm/l to 200 mOsm/l or in the range built up from any of the before-mentioned osmolarities (such as 10 mOsm/l to 150 mOsm/l etc.). In an embodiment, the osmolarity of the aqueous medium is in a range between 0 mOsm/l and 49 mOsm/l, between 0 mOsm/l and 45 mOsm/l, between 0 mOsm/l and 40 mOsm/l, in particular between 0 mOsm/l and 35 mOsm/l, in particular between 0 mOsm/l and 30 mOsm/l, between 0 mOsm/l and 25 mOsm/l.

In specific embodiments, the liposomes can optionally be extruded or filtered to obtain liposomes having a specific size.

The hydration of the lipid bilayer components/lipid blend can be conducted at room temperature (i.e., around 20-25° C.) or while heating (e.g., at a temperature up to 60° C. (e.g., prewarmed aqueous medium), preferably up to 45° C.) and optionally slowly stirring for a period of about 15 minutes to 4 hours, preferably about 2 hours. At that stage, the final concentration of lipids is preferably at about 100 mg/g, if the hydration was performed while heating the mixture is cooled down to room temperature (i.e., around 20-25° C.). The mixture can optionally be degassed (e.g., under vacuum) to remove air bubbles.

In an embodiment, the hydrated liposomes so prepared are sterilized so as to obtain sterilized liposomes or sterilized suspension containing the liposomes. The sterilization can be carried out by, e.g., sterile filtration or steam sterilized (e.g., autoclaving), e.g., for a period of about 5 minutes to 2 hours, 10 minutes to 1 hour, or 15 minutes, or 30 minutes.

In another embodiment, the vesicles are stored for a first period of time prior to carrying out the step of mixing the liposomes (or the liposomes-containing suspension) with the acidic buffer. This storage can be optimally accomplished in if the liposomes are sterilized after the hydration in aqueous medium step because then no or little degradation processes will occur in the sterilized liposomes suspension. The first period of time can be one day, a few days, one week, several weeks (1, 2, 3 or 4 weeks), one month or even several months (e.g., 1, 2, 3, 4, 5, 6, 7, 8, 9, 10, 11 or 12 months). Sterilized liposomes contained in an aqueous medium are stable entities. Since they do not yet contain any specific basic buffer used to prepare the pH gradient, no buffer loss due to liposomes bilayer degradation or leakage of the liposomes has to be feared. This is also true if the liposomes, in an embodiment, contain low amounts of electrolytes molecules since an according osmolarity within the vesicles would then be in a range of between 0 or 1 mOsm/l to 200 mOsm/l.

### Acidic and Hyperosmotic Buffer

Thereafter, the hydrated (and optionally sterilized) liposomes are mixed with an acidic buffer having an osmolarity being at least 200 mOsm/l higher than the osmolarity of the aqueous medium to apply an osmotic shock to the liposomes and to obtain buffer-filled liposomes. In an embodiment, the osmolarity of the acidic buffer is at least 220 mOsm/l higher than the osmolarity of the aqueous medium, at least 250 mOsm/l higher, at least 300 mOsm/l higher, at least 350 mOsm/l higher, at least 400 mOsm/l higher, at least 450 mOsm/l higher, at least 500 mOsm/l higher, at least 550 mOsm/l higher, at least 600 mOsm/l higher, at least 650 mOsm/l higher, at least 700 mOsm/l higher, at least 750 mOsm/l higher, at least 800 mOsm/l higher, at least 850 mOsm/l higher, at least 900 mOsm/l higher, at least 950 mOsm/l higher, at least 1000 mOsm/l higher, at least 1050 mOsm/l higher, at least 1100 mOsm/l higher or at least 1200 mOsm/l higher. In an embodiment, the osmolarity of the acidic buffer is in a range of 200 mOsm/l to 1100 mOsm/l higher than the osmolarity of the aqueous medium or in a range built up from any of the before-mentioned osmolarities (such as 220 mOsm/l to 1200 mOsm/l etc.).

Thus, the acidic buffer is a hyperosmotic buffer with respect to the aqueous medium used in the liposomes hydration step In doing so, an osmotic shock is extemporaneously applied to the liposomes. This osmotic shock results in incorporating the acidic buffer within the liposomes. Thus, the osmotic shock serves for a short-term destabilization of the liposomes in order to allow buffer incorporation into the liposomes. Buffer-filled liposomes result. In an embodiment, the hyperosmotic buffer can also contain electrolytes that are used to modulate the osmolarity or have a physiological function.

A sufficient amount of the acidic buffer is to be added to the liposomes suspended in the aqueous medium since otherwise no osmotic shock will be achieved. A sufficient amount can be, depending on the difference between the osmolarity of the aqueous medium and the osmolarity of the basic buffer, a volume that corresponds to at least 0.1 times the volume of the aqueous medium, at least 0.3 times, at least 0.5 times, at least 0.8 times, at least 1.5 times, at least 2 times, at least 2.5 times, at least 3 times or at least 5 times. In an embodiment, the acidic buffer can be added in a volume that equals the volume of the aqueous medium. In an embodiment, the volume of the acidic buffer to be added can be 0.1 times to 5 times the volume of the aqueous liposome suspension or any other range that can be built up from the before-mentioned values (such as 0.3 times to 3 times, etc.).

In an embodiment, the pH value of the hyperosmotic buffer is in a range of pH 1 to pH 6.9, pH 1.5 to pH 6.5, pH 1.5 to pH 6.0, pH 1.5 to pH 5.5, pH 1.5 to pH 5.0, pH 1.5 to pH 4.5, pH 1.5 to pH 4.0, pH 1.5 to pH 3.5, pH 1.5 to pH 3.0, pH 1.5 to pH 2.5, pH 1.5 to pH 2.0, pH 2.0 to pH 6.0, pH 2 to pH 5.5, pH 2.0 to pH 5.0, pH 2.0 to pH 4.5, or pH 2.0 to pH 3.5.

In specific embodiments, the hyperosmotic buffer can contain additional chemical agents such as a complexing agent or chelating agent.

In specific embodiments, the hyperosmotic buffer comprises salts such as but not limited to sodium chloride, sodium hydroxide, and/or magnesium chloride.

In specific embodiments where a sterile transmembrane pH gradient liposome is preferred, the acidic buffer is sterilized. In such embodiments, where the hydrated liposomes had also been sterilized before loading the acidic buffer, fully sterile buffer-filled liposomes or a fully sterile suspension containing buffer-filled liposomes are prepared. The sterilization can be carried out by, e.g., sterile filtration or autoclaving.

In an embodiment, the mixture of the aqueous medium and the basic or acidic buffer in which the buffer-filled liposomes are suspended has an osmolarity of at least 200 mOsm/l, of at least 220 mOsm/l, of at least 250 mOsm/l, of at least 300 mOsm/l, of at least 350 mOsm/l, of at least 400 mOsm/l, of at least 450 mOsm/l, of at least 500 mOsm/l, of at least 550 mOsm/l. In an embodiment, the osmolarity is in the range of 200 mOsm/l to 550 mOsm/l or in the range built up from any of the before-mentioned osmolarities (such as 220 mOsm/l to 500 mOsm/l etc.).

The liposomes acidic buffer mixture can optionally be incubated. In specific embodiments, the mixture is stirred (e.g., by orbital shaking) at e.g., room temperature.

### Neutralizing Aqueous Solution

Then, a mixture of the aqueous medium and the acidic buffer containing the buffer-filled liposomes is diluted by adding a neutralizing aqueous solution. The mixture of acidic buffer and neutralizing solution makes up a suspension buffer. Thus, after dilution, transmembrane pH-gradient liposomes suspended in the suspension buffer result. Thereby, the pH of the suspension buffer differs from the acidic buffer contained in the buffer-filled liposomes. The pH difference is in an embodiment at least 1 pH unit, at least 1.5 pH units, at least 2 pH units, at least 2.5 pH units, at least 3 pH units, at least 3.5 pH units, at least 4 pH units, at least 4.5 pH unit, at least 5 pH units, at least 5.5 pH units, at least 6 pH units, at least 6.5 pH units, or at least 7 pH units.

In an embodiment, the pH value of the neutralizing solution is in a range of pH 7.1 to pH 14, pH 7.1 to pH 13.5, pH 7.1 to pH 13.0, pH 7.1 to pH 12.5, pH 7.1 to pH 12, pH 7.1 to pH 11.5, pH 7.1 to pH 11.0, pH 7.1 to pH 10.5, pH 7.1 to pH 10, pH 7.1 to pH 9.5, pH 7.1 to pH 9.0, pH 7.1 to pH 8.5, pH 7.2 to pH 14, pH 7.2 to pH 13.5, pH 7.2 to pH 13, pH 7.2 to pH 12.5, pH 7.2 to pH 12, pH 7.2 to pH 11.5, pH 7.2 to pH 11, pH 7.2 to pH 10.5, pH 7.2 to pH 10, pH 7.2 to pH 9.5, pH 7.2 to pH 9, pH 7.2 to pH 8.5, pH 7.3 to pH 14, pH 7.3 to pH 13.5, pH 7.3 to pH 13, pH 7.3 to pH 12.5, pH 7.3 to pH 12, pH 7.3 to pH 11.5, pH 7.3 to pH 11, pH 7.3 to pH 10.5, pH 7.3 to pH 10, pH 7.3 to pH 9.5, pH 7.3 to pH 9, pH 7.3 to pH 8.5, pH 7.4 to pH 14, pH 7.4 to pH 13.5, pH 7.4 to pH 13, pH 7.4 to pH 12.5, pH 7.4 to pH 12, pH 7.4 to pH 11.5, pH 7.4 to pH 11, pH 7.4 to pH 10.5, pH 7.4 to pH 10, pH 7.4 to pH 9.5, pH 7.4 to pH 9, pH 7.4 to pH 8.5, pH 7.5 to pH 14, pH 7.5 to pH 13.5, pH 7.5 to pH 13, pH 7.5 to pH 12.5, pH 7.5 to pH 12, pH 7.5 to pH 11.5, pH 7.5 to pH 11, pH 7.5 to pH 10.5, pH 7.5 to pH 10, pH 7.5 to pH 9.5, pH 7.5 to pH 9, pH 7.5 to pH 8.5, pH 8.0 to pH 13.0, pH 8.5 to pH 12.5, pH 9.0 to pH 13, pH 9.0 to pH 12.5, pH 9.0 to pH 12.0, pH 9.5 to pH 11.5, pH 10 to pH 13, pH 10 to pH 12.5, pH 10 to pH 12.0, pH 10 to pH 11.5, pH 10 to pH 11, pH 10 to pH 12.5, pH 10.5 to pH 12.0, pH 10.5 to pH 13, pH 10.5 to pH 12.5, pH 10.5 to pH 12.0, pH 10.5 to pH 11.5, or pH 10.5 to pH 11. In a specific embodiment, the pH of the neutralizing solution is about 12.5.

In an embodiment, the neutralizing solution has a composition that serves for not disrupting the buffer filled vesicles so as to not destabilize these vesicles. It may contain neutralizing species (basic or acidic, such as weak bases or weak acids) but also chemical agents used to adjust the osmolarity and/or provide a physiological function. Calcium salts can be added in the preparation process to counteract the anticoagulant effects of some weak acids (e.g. citric acid). This is in particular importance is the vesicles are to be used in in vivo applications. Sodium hydroxide, sodium salts (such as sodium chloride), potassium chloride, calcium chloride magnesium salts, lactate salts, glycerol, icodextrin, glucose, sorbitol, fructose, amino acids or xylitol can also be used as ingredients of the neutralizing solution. In specific embodiments, the neutralizing solution contains

In an embodiment, the neutralizing solution has an osmolarity of between 250 mOsm/l and 550 mOsm/l, of between 270 and 520 mOsm/l, of between 290 and 500 mOsm/l, of between 300 and 480 mOsm/l, of between 320 and 450 mOsm/l, of between 330 and 420 mOsm/l, of between 350 and 400 mOsm/l, of between 375 and 400 mOsm/l, of between 385 and 400 mOsm/l or of between 390 and 400 mOsm/l.

In an embodiment, the neutralizing solution has an osmolarity which is less than 200 mOsm/l higher or lower than the osmolarity of the mixture containing the buffer-containing vesicles (i.e., the buffer-containing vesicles solution), in particular less than 150 mOsm/l higher or lower, in particular less than 100 mOsm/l higher or lower, in particular less than 50 mOsm/l higher or lower, in particular less than 20 mOsm/l higher or lower, in particular less than 10 mOsm/l higher or lower. In an embodiment, the difference in osmolarity between the neutralizing solution and the mixture containing the buffer-containing vesicles is between 1 mOsm/to 200 mOsm/l, in particular between 10 mOsm/to 150 mOsm/l, in particular between 20 mOsm/to 100 mOsm/l, in particular between 30 mOsm/to 80 mOsm/l, in particular between 40 mOsm/to 60 mOsm/l.

Due to the pH differences between the suspension buffer and the acidic buffer, a transmembrane pH-gradient between the inner part of the liposomes and the surrounding suspension buffer is achieved. The resulting transmembrane pH-gradient can be used in accordance with the present disclosure.

In an embodiment, the pH value of the suspension buffer containing the transmembrane pH-gradient vesicles is in the range of 5.5 to 8.5, of 6.0 to 8.0, of 6.3 to 7.7, of 6.3 to 7.5, of 6.3 to 7.3, of 6.3 to 7.2, of 6.3 to 7.1, of 6.5 to 7.7, of 6.5 to 7.5, of 6.5 to 7.3, of 6.5 to 7.2, of 6.5 to 7.1, of 6.8 to 7.5, of 7.0 to 7.4. Thus, the suspension buffer may have a physiological pH value. In a specific embodiment, the pH value of the suspension buffer is about 6.5.

Osmotic shock methods are also described in EP 3 291 797 to Leroux et al.

### Alternative Methods of Preparing Liposomes

In another embodiment, the method of preparing the transmembrane pH gradient liposomes include the film hydration method. For example, liposomes bilayer membrane components are dissolved in an organic solvent (e.g., dichloromethane: methanol), the organic solvent is then removed (e.g., by rotary evaporation) to form a dried lipid film. The dried lipid can be stored for future use (e.g., under vacuum). The dried lipid can thereafter be hydrated directly in the acidic buffer described above, and the external solution exchanged with a neutral solution as described above. Alternatively, the film hydration method can first be used to form a lipid film which is then hydrated in the aqueous medium as described above.

The aqueous medium loaded liposomes can thereafter be subjected to the osmotic shock step described above to load the acidic buffer therein and be subjected to the neutralization solution step described above to create the transmembrane pH-gradient liposomes suspension.

Alternatives of methods of preparing liposomes are also described in EP 2 882 421 to Leroux et al.

### Route of Administration and Mechanism of Action

The liposomes of the present disclosure are intraperitoneally administered.

The term “intraperitoneally administration” as used herein is meant to be understood as it is commonly understood by the person skill in the art of peritoneal dialysis treatment. For practicing the invention, a pharmaceutically effective amount of the liposome suspension of the disclosure is administered into the peritoneal cavity, e.g., by injection as a single bolus, by continuous infusion or perfusion, e.g., by catheter, such as catheter commonly used for paracentesis.

The liposomes within the cavity and the nearby tissues and organs will take up the ammonia based on the pH gradient across the liposome membrane. The acidic buffer contained within the liposomes possesses a lower pH than the physiological pH in the peritoneal cavity (which is about 7.5 to 8). Hence, ammonia can diffuse through the hydrophobic liposome bilayer in its uncharged state and be then trapped in its protonated (ionized) state (e.g., ammonium) in the inner liposome compartment.

The liposome sequesters ammonia for a prolonged time period and reduces the toxic concentration of the free compound. The ammonia-loaded liposomes in the peritoneal cavity are removed/extracted from the peritoneal cavity with the fluid present therein (dialysate). Intraperitoneal administration and extraction can be performed subsequently and/or simultaneously. Without being so limited, the dialysate can be extracted by passive drainage through a catheter by gravity or pumped out by suction via a pump such as a peristaltic pump used for infusion.

### Diseases

Acute hyperammonemia in a context of IEM is referred to as “HAC” or “acute hyperammonemia episode” which represent the same medical condition. HAC is defined as plasma ammonia levels above 80-100 μmol/L in newborns up to 1 month of age and above 55 μmol/L in older children and adults [Haeberle 2013]. In published longitudinal studies of IEMs, HACs were defined as “compatible clinical symptoms associated with plasma ammonia levels >100 μmol/L” [Kent 2017] or “an episode of acute hyperammonemia defined as a single hospitalization for hyperammonemia” [Enns 2007].

In mammals, the hepatic urea cycle is the main pathway to detoxify ammonia. Ammonia is continuously produced by the breakdown of protein and other nitrogen-containing molecules. Hyperammonemic crisis occurs whenever the load of waste nitrogen exceeds the detoxification capacity.

Hyperammonemia rapidly leads to cerebral edema and the related signs of lethargy, anorexia, hyperventilation or hypoventilation, hypothermia, seizures, neurologic posturing, and coma. These events occur through a variety of mechanisms, mainly resulting in astrocyte swelling due to increased glutamine. The specific role of ammonia, glutamate, and glutamine in cerebral edema is still under investigation but recent data show that excessive ammonia exposure alters several amino acid pathways and neurotransmitter systems, cerebral energy, nitric oxide synthesis, axonal and dendritic growth, signal transduction pathways, as well as K+ and water channels. All these effects may eventually lead to energy deficit, oxidative stress and cell death.

### Etiology

As indicated above, IEM causing HACs comprise a group of hereditary disorders in which a single gene defect results in a clinically significant block of the urea cycle responsible for the metabolic clearance of ammonia from the bloodstream.

As indicated above, HAC associated with IEM is designated “primary hyperammonemia” when it is secondary to inherited defect of any of the enzymes or transporters involved in the urea cycle, defining UCBs, and is designated “secondary hyperammonemia” when enzymes of the urea cycle are inhibited due to accumulating metabolites or substrate deficiencies. The most relevant group of disorders associated with secondary hyperammonemia is called Organic Acidemias (Oas). Regardless of the underlying genetic disorder, the clinical characteristics, outcome, prognosis and treatment of HACs associated with IEM are similar.

### IEMs Underlying Genetic Defects

### UCDs

UCDs are the most common genetic causes of HACs in infants and children. They result from inherited defects in genes encoding any one of the five enzymes, cofactor and two transporters of the Krebs-Henseleit cycle/Urea cycle [Ah Mew 2013]. The five catalytic enzymes are carbamyl phosphate synthetase 1 (CPS1), ornithine transcarbamylase (OTC), argininosuccinic acid synthetase (ASS), argininosuccinic acid lyase (ASL), and arginase (ARG1) (deficiencies respectively abbreviated as follows: CPS1D, OTCD, ASSD, ASLD and ARG1D, and bearing the numbers respective MIMs: 237300, 311250, 215700, 207900, 207800); the cofactor-producing enzyme is N-acetyl glutamate synthetase (NAGS) (MIM No. 237310), the main activator of CPS1; and the two amino acid transporters are ornithine translocase (ORNT1), which induces hyperornithinemia hyperammonemia-homocitrullinuria syndrome (triple H syndrome (HHH) (MIM no. 238970), and citrin.

The urea cycle as a nitrogen clearance system occurs primarily in the human liver and intestine with CPS1 and OTC limited exclusively to those tissues. The enzymes downstream that process citrulline into arginine are ubiquitous in their distribution. As the rate-limiting enzyme in the urea cycle, functional changes in the enzyme CPS1 is expected to have the greatest impact on the cycle function.

OTC deficiency is inherited in an X-linked manner. The other seven urea cycle disorders (deficiencies of CPS1, ASS, ASL, ARG1, NAGS, ORNT1 and citrin) are inherited in an autosomal recessive manner.

### Organic Acidurias/Acidemia

Organic acidurias (Oas) are inherited disorders mainly affecting the breakdown of branched-chain amino acids. Important Oas causing HAC include propionic aciduria/acidemia (PA), methylmalonic acidurias/acidemia (MMA), isovaleric aciduria/acidemia (IVA), Maple syrup urine disease (MSUD), glutaric aciduria type I and multiple carboxylase deficiency. The PA, MMA and MSUD Oas are sometimes referred to as “classical organic acidurias”, since they involve the most common organic acids.

Hyperammonemia in Oas is due to a decrease in acetyl coenzyme A (CoA) and an inhibition of NAGS and CPS1 activities resulting from a toxic accumulation of organic acid metabolites. In these disorders, the HAC is accompanied by a severe metabolic acidosis with a high anion gap and ketonuria. Organic acidurias induce long-term complications in many organs (including the brain, kidneys, heart, bones, and pancreas) but primarily lead to a neurological phenotype.

Although most of the OAs are autosomal recessive disorders, some are X-linked. The diagnosis of hyperammonemia associated with OA is made by plasma ammonia levels, urinary organic acid chromatography and plasma acylcarnitine profile [Savy 2018].

Regardless of the underlying genetic disorder, the clinical characteristics, outcome and prognosis and current treatments of primary and secondary HACs associated with IEM are similar.

Clinical Characteristics of HACs Associated with IEMs

As indicated above, regardless of the underlying genetic disorder, the clinical characteristics/presentation (symptoms) of primary and secondary HACs associated with IEM are similar.

The clinical presentation of patients with HAC caused by IEM may start as early as the first days of life and as late as adulthood by manifestations of the type acute or chronic. Severity tends to be inversely correlated to the subject's age.

Subjects with HAC caused by IEM are clinically characterized by their neurological, psychiatric or hepatic/gastrointestinal symptoms, or a combination of these three categories of symptoms. In the absence of treatment, or in the event of delayed diagnosis, survivors have severe neurological disabilities and seizures. Even in cases of partial deficit, the manifestation of which is more variable and appears later (at any age), there is a risk of sequelae neurological symptoms linked to hyperammonemia and a risk of death. There is a strong correlation between the duration and severity of hyperammonemia and brain damage making a quick diagnosis and appropriate treatment essential to optimizing patient outcomes.

The severity of the symptoms is related to the residual enzymatic activity and the position of the deficient enzyme in the urea cycle. Thus a severe enzyme deficiency or total absence of enzymatic activity is responsible for an accumulation of ammonia and other metabolites from the first days of life, while a partial deficiency can lead to hyperammonemia of varying intensity often triggered by catabolic events, protein overload or certain medications, regardless of age. Careful verification of medical and family history should be systematic. This includes addressing the following issues: unexplained deaths; presence of disorders psychiatric or neurological in the family; inbreeding (frequent in all UCDs, except OTCD, which is linked to the X chromosome); demonstrated spontaneous avoidance of proteins by the patient and/or family members; medication taken by the patient.

Clinical laboratory data generally useful in the diagnosis of hyperammonemia associated with UCDs include plasma ammonia levels, pH, CO₂, the anion gap, plasma amino acids, and urine organic acid analyses.

General acute presentation. Altered level of consciousness (ranging from drowsiness/lethargy to coma) reminiscent of encephalitis or drug intoxication; acute encephalopathy; seizures (usually accompanied by altered level of consciousness and not isolated); ataxia (usually associated with impaired level of consciousness); pseudo-stroke episodes; temporary loss of sight; vomiting and progressive loss of appetite; hepatic insufficiency; failure of several organs; peripheral circulation disorders; postpartum psychosis; dysarthria; asterixis (in adults); learning disabilities, neurodevelopmental delay, mental retardation; chorea, cerebral palsy; prolonged cortical blindness; progressive spastic diplegia or quadriplegia (described in ARGD1 or triple H syndrome); aversion to protein, voluntary low protein diet; abdominal pain, vomiting; growth retardation; hepatomegaly, elevated liver enzymes; migraine-like headache, tremors, ataxia, psychiatric symptoms (hyperactivity, altered mood, altered behavior, aggressiveness, hallucinations, paranoia, manic episodes, emotional disturbances and altered personality); self-endangerment; pseudo-autistic symptoms; fragile hair (characteristic of ASLD); specific neuropsychological phenotype in heterozygous OTC patients; episodic nature of signs and symptoms.

Neonatal presentation. Affected newborns usually have no symptoms at birth. After an interval free from 24 hours to a few days, they will quickly present irritability that progress rapidly to somnolence/drowsiness, anorexia/feeding refusal and vomiting, loss of thermoregulation, neurologic posturing, cerebral edema resulting in lethargy, hyperventilation and then hypoventilation, hypothermia, hypotonia or hypertonia, convulsions/seizures, coma, multi-organ failure and death [Ah Mew 2013; Summar 2008; Haeberle 2013]. These symptoms can mimic an array of sepsis which can be a source of delay in diagnosis. The clinical presentation of HACs associated with the milder forms of IEM having residual enzyme activity usually occurs later in life (late-onset at almost any time from infancy to adulthood) with recurrent episodes of mild to moderately severe hyperammonemia potentially triggered by illness, stress, or by excessive protein intake [Summar 2008]. In late-onset IEM, HACs are usually less severe and the symptoms more subtle.

It was reported that only 27% of 678 UCD patients enrolled in the UCDC Natural History study had a neonatal HAC. Similarly, Summar reported that the majority (66%) of 260 patients with UCD present with HACs beyond the neonatal period (>30 days) (FIG. 1A).

Adult presentation. Adults (aged over 16 years) with UCD deficiency either come from the pediatric cohort or are diagnosed later (any possible age, youth or old age). In both cases, the clinical manifestations are the same as those of the infantile or juvenile form.

The diagnosis of UCD in adulthood is usually made during acute decompensation. These are mostly OTC deficiencies and mostly women. The clinical signs are mainly neurological, dominated by disorders of consciousness and psychiatric symptoms. Only a small percentage of patients have never shown prior signs suggestive of decompensation and two-thirds have an aversion to animal proteins and a spontaneously vegetarian diet.

Predisposing factors for HAC in UCD patients. Infections; fever; vomiting, diarrhea; internal or gastrointestinal bleeding; decreased protein or energy intake (e.g., preoperative fasting, major weight loss in newborns); catabolism and involution of the uterus during the postpartum period (mainly in OTC patients); chemotherapy, high doses of glucocorticoids; intense or prolonged physical exercises; surgery under general anesthesia; excessive protein intake (e.g., protein-rich foods: meat, fish, eggs, dairy products; unsuitable artificial nutrition). Drugs: mainly valproate and L-asparaginase/pegaspargase. Topiramate, carbamazepine, phenobarbitone, phenytoin, primidone, furosemide, hydrochlorothiazide and salicylates have also been associated with hyperammonemic decompensations; a little more specific for adults: rapid weight loss (weight loss diets, bariatric surgery) anorexia, postpartum.

### Outcome and Prognosis

An early age of onset (FIG. 1B) and a severe clinical presentation (coma and high plasma ammonium levels >1000 μmol/L) (FIG. 1C) correlate with high mortality rates and a poor neurological prognosis [Krivitzky 2009; Enns 2007]. It was estimated that until recently, approximately 25% of neonates with high exposure to ammonia caused by UCDs die prematurely and this percentage continues to increase (FIG. 1D) [Hediger 2018].

Historically, the majority of children who manifested an early onset of neonatal HAC due to a severe UCD or OA enzyme deficiency died as neonates, and few survived infancy. Although later onset of HAC related to IEM is usually associated with a better outcome [Enns 2007; Hediger 2018], neurologic morbidity remains high, with a significant proportion of patients showing poor neurocognitive outcome and behavioral impairment [Krivitzky 2009; Ah Mew 2013]. Krivitzky reported that approximately half of the children with neonatal onset of HAC showed intellectual disability, among whom 30% were severely impaired. In comparison, only a quarter of the late onset group were affected by intellectual disability despite evidence of neurocognitive and behavioral impairment related to attention and executive functioning (FIG. 1B).

Published data from 103 subjects with neonatal onset of HAC derived from the Longitudinal Study of UCDs Showed that across all types of enzyme defects, between 47% and 68% of pediatric patients had a poor outcome (IQ/Developmental Quotient <70). This was observed in both patients <4 years of age and those ≥4 years of age and did not differ between patients with proximal UCDs (FIG. 1E) and those with distal UCDs (i.e., ASS and ASL).

### Combination Therapy

The present disclosure encompasses combining an intraperitoneal administration of the transmembrane pH-gradient liposomes as described herein with other therapies for treating HAC associated with IEM (simultaneously or sequentially (e.g., presently disclosed emergency treatment first following by at least one other treatment), depending on the nature of the additional treatment) for acute or chronic treatment.

### Treatment and Prevention

The present disclosure encompasses the use of the liposomes/liposome suspension or composition as described herein for the treatment of an acute hyperammonemia episode (HAC) associated with inborn errors of metabolism (IEM) in a subject. It may further comprise the treatment or prevention of any symptom of HAC associated with IEM downstream of acute hyperammonemia per se as further described herein.

The terms “treat/treating/treatment” as used herein, refers to eliciting the desired biological response, i.e., a therapeutic effect. In accordance with the disclosure herein, the therapeutic effect comprises one or more of a decrease/reduction in the frequency, duration and/or severity of HAC associated with IEM. It may further comprise one or more of a decrease/reduction of frequency, duration and/or severity of at least one a symptom triggered by the HAC, and/or duration of symptom-free periods following administration of the liposomes of the present disclosure as described herein, or of a composition (e.g., suspension) comprising the liposomes of the present disclosure, alone or in combination with another agent for the treatment of a HAC associated with IEM or at least one symptom thereof.

The terms “prevent/preventing/prevention” as used herein, refers to eliciting the desired biological response, i.e., a prophylactic effect. In accordance with the disclosure provided herein, in some embodiments, a prophylactic effect comprises a complete or partial avoidance/inhibition of at least one symptom of HAC associated with IEM following administration of the liposomes or liposomes suspension or composition of the present disclosure, alone or in combination with another agent for the prevention or treatment of a muscular dystrophy or of at least one a symptom thereof.

In some embodiments, “therapeutically effective amount” or “effective amount” or “therapeutically effective dosage” of a liposome or liposome suspension of the present disclosure provided herein results in a treatment of HAC associated with IEM in a subject in need thereof. It may also result in the treatment or prevention of at least one a symptom thereof in a subject.

As used herein the term “at least one symptom of HAC associated with IEM” refers to any of the clinical characteristics of the pediatric or adult subjects described under the headings “general acute presentation, “Neonatal presentation” or “adult presentation” and correspond to events resulting at least in part from the acute hyperammonemia per se.

### Subjects

As used herein the terms “subject” or “subject in need thereof” refer to a subject who would benefit from receiving an effective amount of the liposomes and liposome suspension. It refers to an animal, mammal and to a human in a specific embodiment. The compositions of the present invention may also be used for veterinary applications and be used in pets or other animals (e.g., pets such as cats, dogs, horses, etc.; and cattle, fishes, swine, poultry, etc.). In specific embodiments, the subject suffers from HAC associated with IEM. In specific embodiment the subject has a healthy liver and/or does not suffer from drug-induced hyperammonemia. In specific embodiment, the subject is a pediatric subject. As used herein the term “pediatric subject” refers to a subject aged 21 or younger at the time of their diagnosis or treatment: i.e., neonates (i.e. from birth through the first 28 days of life), infants (about 29 days to less than 2 years old), children (from 2 years to less than 12 years old), or adolescents (about 12 years old to 21 years old) with recurrent HACs In another more specific embodiment, the subject is a neonate, an infant or a child. In another specific embodiment, the subject is an adult.

The subject in need thereof is pre-diagnosed as having a HAC associated with IEM. In certain embodiments, the methods of the present disclosure encompass a step of diagnosing the subject

The use of the terms “a” and “an” and “the” and similar referents in the context of describing the disclosure (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

All methods described herein can be performed in any suitable order unless otherwise indicated herein or otherwise clearly contradicted by context.

The use of any and all examples, or exemplary language (e.g., “such as”) provided herein, is intended merely to better illuminate the disclosure and does not pose a limitation on the scope of the disclosure unless otherwise claimed.

No language in the specification should be construed as indicating any non-claimed element as essential to the practice of the disclosure.

Herein, the term “about” has its ordinary meaning. In embodiments, it may mean plus or minus 10% of the numerical value qualified.

Unless otherwise defined, all technical and scientific terms used herein have the same meaning as commonly understood by one of ordinary skill in the art to which this disclosure belongs.

Other objects, advantages and features of the present disclosure will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1A: Number of patients by diagnosis and age at first episode. Each patient is counted once at the age of the first episode of hyperammonemia was reported [Summar 2008].

FIG. 1B: Cognitive range across UCD subjects ages 3-6: neonatal (NO) vs. Late Onset (LO) [Krivitzky 2009].

FIG. 1C: The percentages of episodes of hyperammonemia survived according to peak ammonium levels was based on the number of episodes for which data on plasma ammonium levels were available [Enns 2007].

FIG. 1D: Relation between main outcome parameters classified as “normal outcome”, “deceased”, “handicapped”, and “alive but with no further information” (alive-no-info) specified for single and groups UCDs. Absolute numbers are given above each column [Hediger 2018].

FIG. 1E: Neurodevelopmental outcome of subjects aged 4 years and older by diagnosis [Ah Mew 2013]. Each diagnostic cohort, proximal UCD (CPSD/OTCD), ASD, ALD, as well as the total neonatal UCD cohort, is stratified by neurodevelopmental outcome. When no Full-Scale Intelligence Quotient (FSIQ) was available, Verbal IQ or Performance IQ was used to determine categorization. Profound/severe range of disability, Subject was not testable by traditional IQ testing for their age range Bayley Scales were instead administered to derive a DQ; Mild-moderate range of disability, FSIQ score of 45-69. Low average/borderline functioning, FSIQ score of 70-89; Broadly average, FSIQ score 90-109; Above average, FSIQ score 110 (No subject met this criterion).

FIG. 2: Linear regression model for the estimation of the relationship between Ammonia Clearance in Peritoneal Fluid (mL/min) and volume VS-01 Infused (mL).

FIG. 3: Mean Peritoneal Fluid Ammonia Concentrations in B6EiC3Sn a/A-Otcspf-ash/J Mice Following a Single Session with VS-01; Sidak's multiple comparisons test.

FIGS. 4A-B. Mean Blood Ammonia Concentrations in B6EiC3Sn a/A-Otcspf-ash/J Mice Following a Single Session with VS-01. FIG. 4B reproduces the graphs of FIG. 4A but includes ammonia concentration achieved with VS-01 at 1 hr postdose for each of the four mice separately (i.e., 2005 (outlier), 2006, 2007 and 2008) in addition to the average value for all mice of the VS-01 Group. Dunnett's multiple comparisons test.

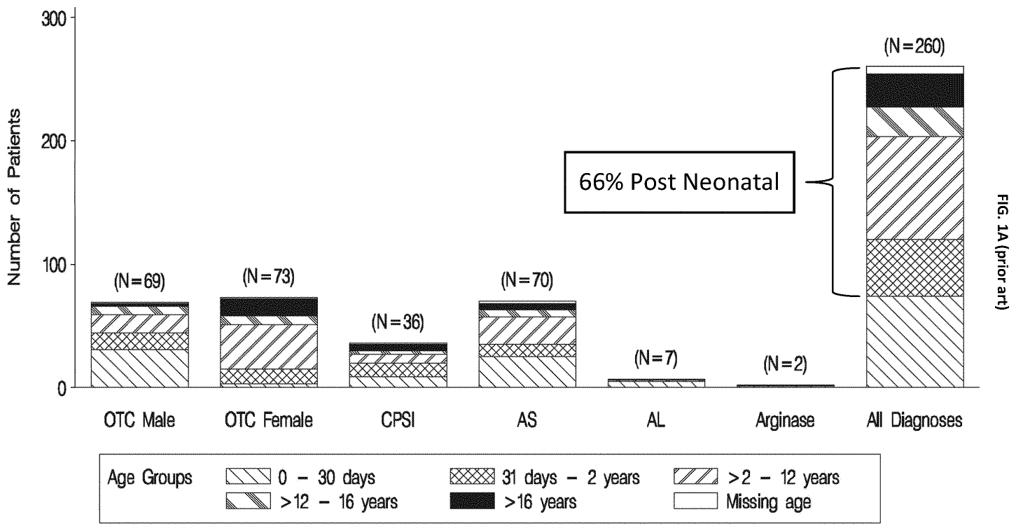

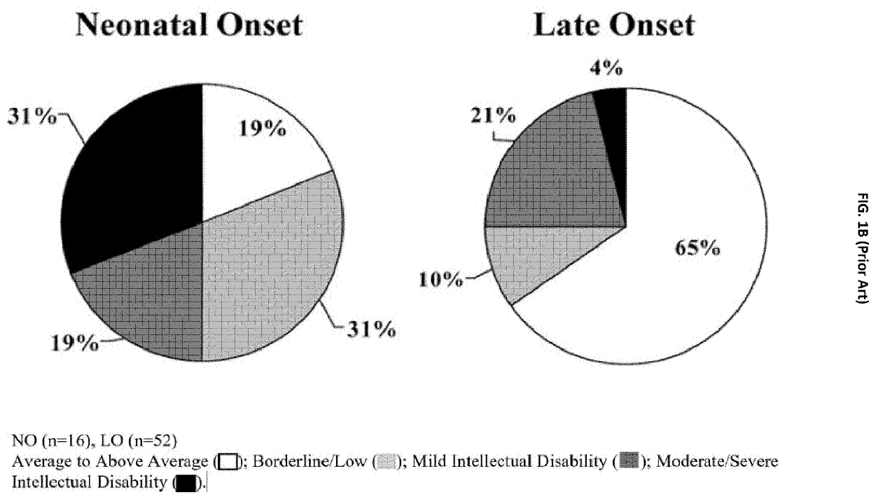

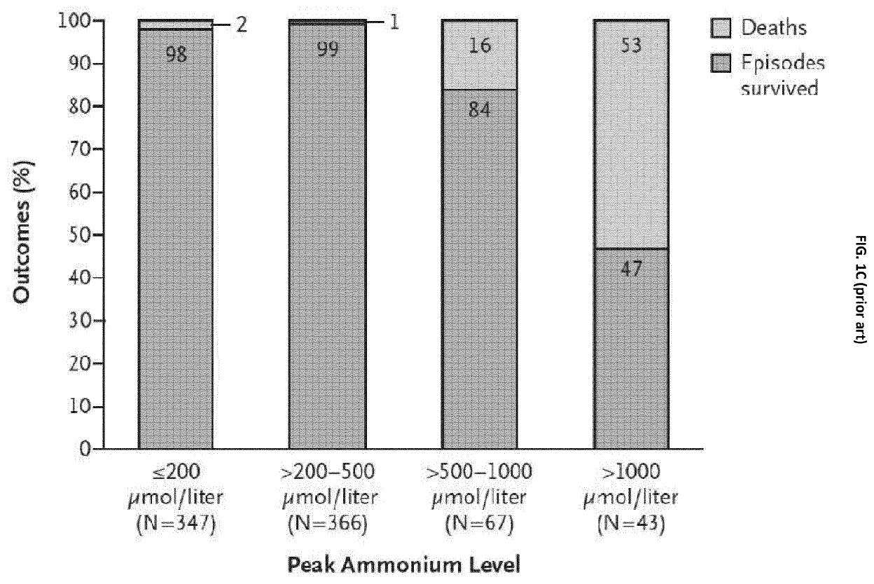

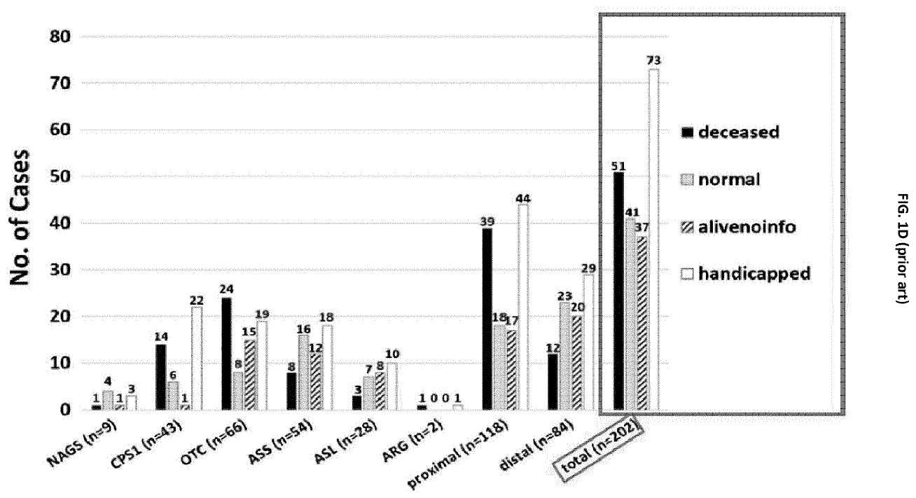

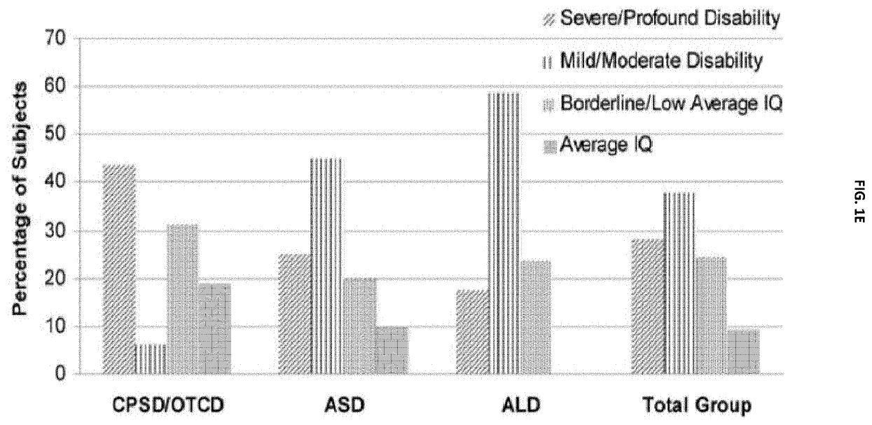

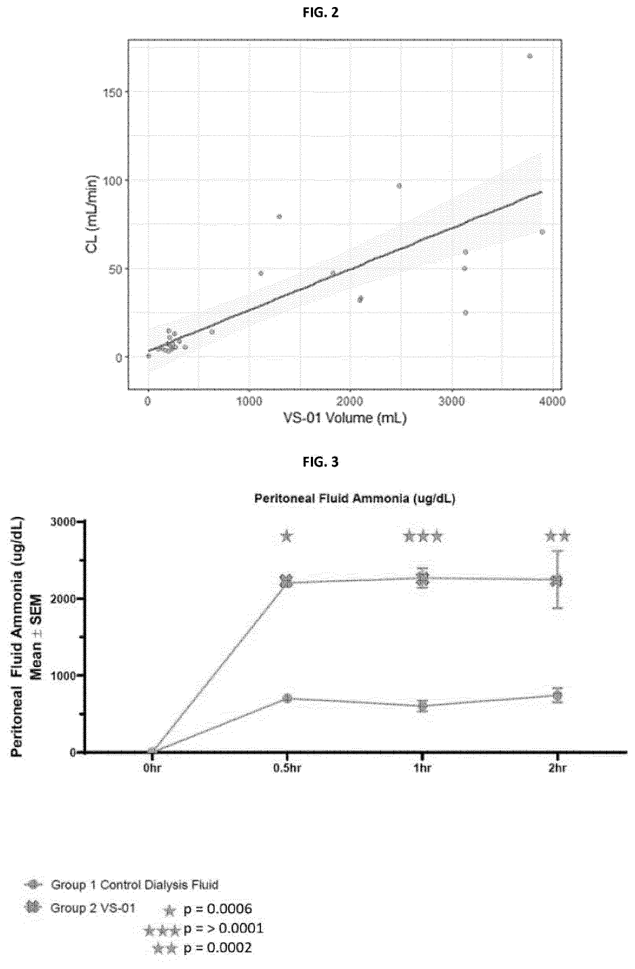

### DESCRIPTION OF ILLUSTRATIVE EMBODIMENTS

The present disclosure is illustrated in further details by the following non-limiting examples.

### Example 1: Material and Methods

### Mice

Hemizygous males B6EiC3Sn a/A-Otc^{spf}-ash/J mice, strain #: 002343. RRID: IMSR_JAX: 002343 (common name: sparse fur), were used in the assays. These mice, characterized by late and patchy development of fur, carry the spontaneous Otc^{spf} (sparse fur) mutation on the X chromosome, a C to A missense transversion mutation in exon 4 which changes a histidine to an asparagine (H117N), creating a hypomorphic allele. This mutation results in only 5-10% normal hepatic ornithine transcarbamylase (OTC) activity ultimately resulting in higher plasma concentration of ammonia. This deficiency of liver OTC is similar to that found in congenital hyperammonemia type II seen in children, characterized by life-threatening episodes of acute metabolic decompensation with hyperammonemia.

### Liposome Solution Formulation

Liposomes composed of dipalmitoylphosphatidylcholine (DPPC, Lipoid), cholesterol (Sigma-Aldrich) and 1,2-distearoyl-sn-glycero-3-phosphoethanol-amine-N-[methoxy(PEG)-2000] (DSPE-PEG, Lipoid) at 85.5:14:0.5 mol % were prepared by the direct lipid hydration method. 595.4 mg of DPPC, 51.3 mg of cholesterol and 13.3 mg of DSPE-PEG were co-dissolved in a 70:30 w/w t-butanol:water mixture to form a homogenous mix. The organic solvent was subsequently removed by lyophilization. The dried lipid blend was hydrated with ultra-pure water (lipids concentration=100 mg/mL) while heating and slowly mixing 2 hours at 60° C. and finally sterilized in sealed bottles by autoclaving 15 min at 121° C. Liposomes having an average diameter between about 8 μm and 12 μm were obtained.

68.1 mL of the so-obtained liposomes were incubated 30 min with 32.2 mL of citrate buffer 600 mM (pH 2.1, 1041 mOsm/l) containing citric acid, sodium chloride, sodium hydroxide, and magnesium chloride. The incubation was performed under orbital shaking, at room temperature. The resulting liposomes contained 200 mM citric acid and had an internal pH of about 2.

The transmembrane pH-gradient was generated by neutralizing the external acid medium with 950 ml of neutralization solution (pH=12.5, 392 mOsm/l) made of xylitol, sodium chloride, sodium hydroxide, potassium chloride, and calcium chloride. The resulting multilamellar liposomes suspension containing 18.4 mM citric acid anhydrous, at pH 6.5 and about 310 mOsm/l was used for the preclinical studies.

### Study Design:

This non-clinical study was performed according to good scientific principles and internal standard operating procedures of Charles River Laboratories, Inc., MA 01545 USA.

Twenty-two male B6EiC3Sn a/A-Otc^{spf}-ash/J mice (˜7-8 weeks of age at dose initiation) were received from The Jackson Laboratory. Animals were single housed in polycarbonate cages which contained appropriate bedding. Upon arrival, all animals were fed LabDiet™ 5LG4 and hydrogel pellets ad libitum except during designated procedures. Municipal tap water after treatment by reverse osmosis and ultraviolet irradiation was freely available to all animals. On Day −1, all animals were weighed, given a blood ammonia reading via handheld meter (ARKRAY PocketChem BA) and assigned to treatment groups in such a way as to generate groups with no significant differences with regards to bodyweight and blood ammonia. Treatment was initiated under fasted conditions on Day 1.

On the day of the experiment, liposomes solution VS-01 was reconstituted under a laminar flow hood using clean procedures. A commercial dialysis fluid (Dianeal) was used as a control article.

A volume of 100 ml/kg of both Dianeal (control) and liposomes solution (VS-01) (354 mg/kg/day citric acid) were administered via an intraperitoneal injection to Group 1 and Group 2 animals, respectively. The solutions were left in the abdominal cavity over a dwell time of 0.5, 1 and to 2 hours according to the assigned sampling timepoint as described below.

On Day 1 following 5 hours fasting, whole blood from a submandibular collection was tested for ammonia levels at the following timepoints: pre-dose in all groups, then in a staggered fashion following dosing, with 3 animals from Group 1 tested at 0.5, 1 and 2 hours postdose, 4 animals from Group 2 tested at 0.5 and 1 hour post-dose, and 5 animals from Group 2 tested at 2 hours post-dose.

Following the same schedule, peritoneal dialysate samples were collected +/−10 min via intraperitoneal aspiration (21 g needle/1 ml syringe), transferred into a uniquely labeled cryovial, and frozen on dry ice. All samples were stored within 1 hour in a freezer set to maintain −80° C. pending analysis using a validated ammonia method [Cobas 6000/NH₃L2 Ammonia Assay from Roche Diagnostics (MLM-VAL-1954)]. Study parameters included mortality, cage side observations and body weight measurements. Animals were euthanized via CO₂ asphyxiation on Day 1.

### Example 2: Ammonia Clearance in the Peritoneal Fluid of UCD Model Significantly Increased with Volume of VS-01 Administered

All animals tolerated dosing well with no signs of adverse reactions to the treatment.

Ammonia extracted from blood into the peritoneal cavity was significantly (p<0.0006) higher following single intraperitoneal injection of the liposomal suspension of Example 1 (VS-01) (354 mg/kg/day citric acid) compared to the control solution at all timepoints during the dwell time (FIG. 3).

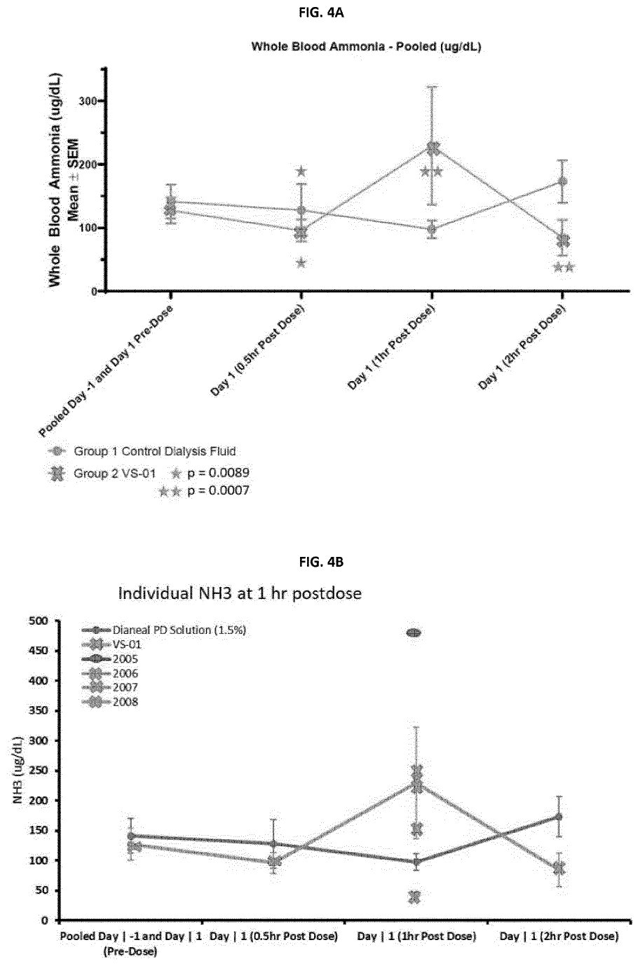

This led to a significant decrease in blood ammonia at 0.5 hour (p<0.0089) and 2 hours (p<0.0007) postdose (FIG. 4A). At 1 hour postdose, mean blood ammonia was skewed by an outlier value in animal 2005 likely related to the injection procedure which may have increased systemic exposure to VS-01 (FIG. 4B).

The peritoneal fluid ammonia clearance in OTC mice was calculated from blood exposure to ammonia over the dwell time of 2 hours (AUC0-2) and the amount of ammonia extracted at 2 hours postdose (Amount=Concentration in peritoneal fluid×Volume fluid injected). The mean value of ammonia clearance in the peritoneal fluid was 0.3 mL/min versus 0.1 mL/min following VS-01 and Dianeal treatment, respectively.

The foregoing shows that VS-01 is useful to treat HAC associated with IEM.

The scope of the claims should not be limited by the embodiments set forth in the examples, but should be given the broadest interpretation consistent with the description as a whole.

### REFERENCES

Ah Mew 2013 Clinical Outcomes of Neonatal Onset Proximal versus Distal Urea Cycle Disorders Do Not Differ. The Journal of Pediatrics.
Enns 2007 Survival after Treatment with Phenylacetate and Benzoate for Urea-Cycle Disorders N Engl J Med 2007; 356:2282-92.
Haeberle 2013 Clinical and biochemical aspects of primary and secondary hyperammonemic disorders Archives of Biochemistry and Biophysics 536 (2013) 101-108.
Hediger 2018 The impact of ammonia levels and dialysis on outcome in 202 patients with neonatal onset urea cycle disorders J Inherit Metab Dis.
Horslen 2003 Isolated Hepatocyte Transplantation in an Infant With a Severe Urea Cycle Disorder Pediatrics 2003; 111:1262-1267.
Kent 2017 Hyperammonemic crises in patients with urea cycle disorders on chronic nitrogen scavenger therapy with either sodium phenylbutyrate or glycerol phenylbutyrate Neuropsychiatry (London) (2017) 7 (2), 131-136.
Krivitzky 2009 Intellectual, Adaptive, and Behavioral Functioning in Children with Urea Cycle Disorders Pediatr Res. 2009 July; 66 (1): 96-101.
Leonard 2004 The role of liver transplantation in urea cycle disorders Molecular Genetics and Metabolism 81 (2004) S74-S78.
Raper 2003 Fatal systemic inflammatory response syndrome in a ornithine transcarbamylase deficient patient following adenoviral gene transfer. Molecular Genetics and Metabolism 80 (2003) 148-158.
Savy 2018 Acute pediatric hyperammonemia: current diagnosis and management strategies. Hepatic Medicine: Evidence and Research 2018:10 105-115.
Summar 2008 Diagnosis, Symptoms, Frequency and Mortality of 260 Patients with Urea Cycle Disorders from a 21-Year, Multicentre Study of Acute Hyperammonaemic Episodes Acta Paediatr. 2008 October; 97 (10): 1420-1425.

1. A method of treating an acute hyperammonemia episode (HAC) associated with inborn errors of metabolism (IEM) in a subject comprising: (a) intraperitoneally administering a therapeutically effective amount of liposomal suspension comprising transmembrane pH-gradient liposomes to the subject; and (b) removing a dialysate containing the liposomes from the subject, wherein the liposomes are ammonia-loaded.

2. The method of claim 1, wherein the subject is a pediatric subject.

3. The method of claim 1, wherein the subject has a urea cycle disorder (UCD).

4. The method of claim 3, wherein the UCD is ornithine transcarbamylase deficiency.

5. The method of claim 1, wherein the liposomes contain a hydroxy acid, preferably citric acid, most preferably citric acid anhydrous.

6. The method of claim 5, wherein the liposomes contain about 200 nM citric acid anhydrous.

7. The method of claim 1, wherein the liposomes' lipid bilayer comprises at least one phospholipid as main constituent.

8. The method of claim 7, wherein the at least one phospholipid comprises dipalmitoylphosphatidylcholine (DPPC), preferably in a range of 60 mol % to 90 mol %.

9. The method of claim 1, wherein the liposomes' lipid bilayer comprises cholesterol, preferably in a range of 10 to 40 mol %.

10. The method of claim 9, wherein the liposomes' lipid bilayer further comprises 1,2-distearoyl-sn-glycero-3-phosphoethanol-amine-N-[methoxy(PEG)-2000] (DSPE-PEG), preferably in a range of 0.2 to 5 mol %.

11. The method of claim 1, wherein the liposomes' lipid bilayer contains dipalmitoylphosphatidylcholine (DPPC), cholesterol and 1,2-distearoyl-sn-glycero-3-phosphoethanol-amine-N-[methoxy(PEG)-2000] (DSPE-PEG) at 85.5:14:0.5 mol %, and the liposomes' inner compartment contains citric acid anhydrous.

12. The method of claim 1, wherein the liposomes have an average diameter between about 8 μm and 12 μm.

13. The method of claim 1, wherein the liposomal suspension contains (i) xylitol, (ii) sodium chloride, (iii) sodium hydroxide, (iv) potassium chloride, (v) calcium chloride or (vii) any combination of at least two of (i) to (v), preferably the combination comprises all of (i) to (v).
